(19) United States
(12) Patent Application Publication
(10) Pub. No.: US 2025/0249091 A1
(43) Pub. Date: Aug. 7, 2025

(54) TREATMENT OF ALLERGY WITH DIMERIC IgA or SECRETORY IgA ANTIGEN IMMUNE COMPLEXES

(71) Applicant: Michael R. Simon, Ann Arbor, MI (US)

(72) Inventor: Michael R. Simon, Ann Arbor, MI (US)

(21) Appl. No.: 19/042,254

(22) Filed: Jan. 31, 2025

Related U.S. Application Data
(60) Provisional application No. 63/549,009, filed on Feb. 2, 2024.

(51) Int. Cl. A61K 39/35; A61K 47/68; A61P 37/08

(52) U.S. Cl. CPC A61K 39/35; A61K 47/6811; A61P 37/08

(57) ABSTRACT

A composition is provided that includes dimeric IgA or secretory IgA that is complexed with a first preselected IgE mediated antigen protein. A method for treating a subject suffering from IgE mediated allergy is provided that includes administering the composition to a subject suffering from an allergy or sensitivity to the preselected IgE mediated antigen protein. The subject is readily treated simultaneously with more than one type of composition that vary in that the dimeric IgA or secretory IgA to complexed to a second a preselected IgE mediated antigen protein.

### RELATED APPLICATIONS

This invention claims priority of U.S. Provisional Application Ser. No. 63/549,009, filed Feb. 2, 2024; the contents of which are hereby incorporated by reference.

### FIELD OF THE INVENTION

This invention relates in general to a composition of matter and methods for the treatment of allergy, and in particular to orally administered human dimeric IgA (dIgA) or secretory IgA (sIgA) or complexed with antigen(s) (sIgA-ag) or (dIgA-ag), compositions administered in the form of pharmaceutical compositions, and an efficient industrial methods for making the same.

### BACKGROUND OF THE INVENTION

A food allergy is an adverse reaction to food ingestion based on the presence of immunoglobulin E (IgE) specific to food antigens (Granato and Piguet 1986; Wang et al. 2010). The IgE is bound to Fc [symbol] receptors on intestinal and other tissue-bound mast cells and circulating basophils (Ra et al. 1989). After cross linking of a required number of these cell surface receptors by food antigens, the mast cells and/or basophiles are activated and release the mediators of Type I hypersensitivity. These mediators may result in crampy abdominal pain, nausea, vomiting, diarrhea and/or anaphylaxis and death (Sampson H., 2006). Initial treatment of food allergy consists of avoidance of foods and/or beverages that contain the allergenic food protein. Keeping to this diet is challenging, especially when a varied diet is consumed and there is always a concern about trace contamination. It has been recently shown in mice that a specific food antigen that was artificially bound to the antigen binding sites (Fab) of IgA dimers and then administered into the gastrointestinal tract by gavage resulted in down regulation of the T helper cell (T_{H}2) responses that induce and maintain allergic disease. This was accompanied by an increase of regulatory T cells. The result was that the mice were specifically protected from diarrhea and other murine manifestations of allergic anaphylaxis when the mice were fed the food that was bound by the IgA dimers (Elesela et al. 2023). Secretory IgA was not used.

Gastrointestinal symptoms in patients with symptomatic food allergy who adhere to a strict avoidance diet typically resolve within several days. However, Food and Drug Administration (FDA) approved treatments for food allergy have provided less than optimal protection (Fleischer et al. 2019, The PALISADE Group, 2018). Oral peanut immunotherapy for peanut allergy (Arachis hypogaea allergen powder) which consists of graded oral administration of progressively larger amounts of food antigen (food challenge) administered weekly is also used. However, food-based challenge treatments are associated with frequent allergy-related adverse events, and more frequent anaphylactic reactions than occurs with either avoidance or placebo (Fleischer et al. 2019,). For this reason, the development of novel therapeutic approaches to combat these reactions has been attempted, yet has met with limited success.

Foods normally induce local intestinal mucosal production of IgA and IgM (Shimoda et al. 1999). Food allergy is associated with deficiency in IgA (Walker et al. 1999, Harrison et al. 1976). It has been hypothesized that food antigen IgA may competitively bind to food antigens, and thereby protect the subject from reacting to that food with an allergic response (Possin et al. 2010). Food antigen specific IgA is found in the blood plasma (Vojdani 2009). Food allergy is associated with a relative decrease food antigen specific IgA in the intestines (Frossard et al. 2004). Application of antigen-specific IgA to the respiratory mucosa in mice prevents increased airway hyperreactivity in allergic asthma (Schwarze et al. 1998; U.S. Pat. No. 5,670,626).

IgE also mediates the immune response to aeroantigens and those associated with dermatological or insect bites' saliva and with Hymenoptera insect sting venom. As a result, IgE is associated with conditions that include allergic asthma, allergic rhinitis, atopic dermatitis, and insect sting allergy (Platt-Mills 2001).

However, the above prior art failed to explore orally administered polyclonal semi-synthetic secretory IgA as a potential medicament, which is prepared using recombinant human secretory component and natural human polyclonal dimeric and polymeric plasma-derived IgA.

Thus, there exists a need for a polymeric IgA therapeutic such as secretory IgA that is resistant to gastrointestinal tract degradation, where the resistance allows oral administration. There also exists a need to provide such a therapeutic in a dosing form well suited for treating an affected subject. There also exists a need for an industrial process for the manufacture of human secretory IgA derived as a byproduct from the industrial processing of pooled donor plasma following the recovery of other plasma proteins.

### SUMMARY OF THE INVENTION

A composition is provided that includes dimeric IgA or secretory IgA that is complexed with a first preselected IgE mediated antigen protein.

A method for treating a subject suffering from IgE mediated allergy is provided that includes administering the composition to a subject suffering from an allergy or sensitivity to the preselected IgE mediated antigen protein. The subject is readily treated simultaneously with more than one type of composition that vary in that the dimeric IgA or secretory IgA to complexed to a second a preselected IgE mediated antigen protein.

A method of producing an inventive composition includes the addition of recombinant human secretory component to dimeric or polymeric IgA to form secretory IgA (sIgA) that then is complexed with a preselected IgE mediated antigen protein (allergen), the composition abbreviated herein as sIgA-allergen. The dimeric or polymeric IgA is readily obtained from polyclonal dimeric or polymeric plasma derived IgA.

One form of the composition includes naturally occurring IgA antibodies that include the secretory IgA therapeutic is dimeric IgA-food allergen immune complexes formed by combining polyclonal dimeric or polymeric IgA containing J chain with or without a recombinant human secretory component. The recombinant human secretory component, if present, is used in a molar ratio of the dimeric or polymeric IgA to the secretory component of 1:1. The secretory IgA food allergen immune complex is formed by combining the dimeric IgA or secretory IgA to the food antigen in a molar ratio of from 4000:1 to 4:1. Formulating agents are readily mixed with the composition to yield a dosing form of a capsule, tablet, and an aerosol. The composition is optionally administered as a solution for oral delivery.

### DETAILED DESCRIPTION OF THE PREFERRED EMBODIMENTS

The present invention has utility as a treatment for IgE mediated allergy or inhibition of food allergen-induced symptoms. The method includes treatment with dimeric and/or polymeric secretory IgA therapeutic alone, or complexed with a preselected allergen having an antigen binding end specific for the antigeen. Because the inventive therapeutic is resistant to degradation in the gastrointestinal tract, dimeric IgA or secretory IgA is administered orally. Dimeric IgA according to the present invention is bound to recombinant human secretory component in order to reconstitute secretory IgA endogenous to the subject. The composition is completed by the binding of the secretory IgA to its target allergen.

It is to be understood that in instances where a range of values are provided that the range is intended to encompass not only the end point values of the range but also intermediate values of the range as explicitly being included within the range and varying by the last significant figure in the range. By way of example, a recited range from 1 to 4 is intended to include 1-2, 1-3, 2-4, 3-4, and 1-4.

As used herein, a “subject” is defined as a mammal and specifically include a human.

As used herein, “dimeric and polymeric IgA” is defined as a construct that contains two or more IgA monomers plus J chain. The dimeric secretory IgA contains 4 antigen binding domains. The polymeric secretory IgA contains more than 4 antigen binding domains.

As used herein, an “IgE mediated antigen” i.e. allergen is defined as an exogeneous protein to the subject that elicits an allergic reaction or a sensitivity through IgE antibodies to the exogenous protein. Exemplary IgE mediated antigens include those found in foods, aeroallergenic eukaryotic cast-offs, topical exposures, and insect stings.

The nomenclature of the World Health Organization/International Union of Immunological Societies (WHO/IUIS) is used as to specific allergenic proteins herein.

As the present invention uses an immunoglobulin rather than a metabolic or immunological inhibitor, an effective treatment is provided which does not disturb the normal metabolism of the subject. An additional advantage of the dimeric and polymeric polyclonal secretory IgA derived from pooled plasma from as many of several thousand donors is that the dimeric and polymeric polyclonal secretory IgA is that it promotes the reconstitution of a diverse microbiome thereby correcting the dysbiosis that is associated with food allergy. Additionally, the natural polyclonal nature of the secretory IgA is that it has antigenic specificity for multiple epitopes on the target molecules.

The following description of the preferred embodiment(s) is merely exemplary in nature and is in no way intended to limit the invention, its application, or uses.

An inventive composition is provided that includes secretory IgA that is complexed with a first preselected allergen protein. In some inventive embodiments, a second preselected allergen protein is also complexed to the polyclonal secretory IgA.

The food antigen protein as selected for complexation to dIgA or sIgA is largely dictated by the food reaction induced in a given subject and by the demonstrated presence in the pooled donor-derived dIgA or secretory IgA of antigen-specific dimeric and polymeric secretory IgA that is specific for the target allergenic food protein(s). The presence of the food antigen-specific pooled donor-derived dIgA or secretory IgA is determined using an enzyme-linked immunosorbant assay (ELISA) as is widely practiced by those with expertise in the state of the art. It is appreciated that a food antigen protein as used herein can include multiple epitopes, various glycosylation sugars, protein fragments of naturally occurring proteins with the proviso that antigenic portions of the naturally occurring are retained therein, and combinations thereof. Specific sources of food antigen proteins illustratively include cow's milk; chicken egg; fish, such as bass, flounder, and cod; crustaceans, such as crab, lobster, crayfish, and shrimp; tree nuts such as cashew, walnut, pistachio, almond, pecan, Brazil nut, pinenut, hazelnut and macadamia nut; peanuts and other legumes such as soybeans and sesame; wheat. Specific food antigen proteins operative in the present invention illustratively include casein, β-lactoglobulin, α-lactalbumin, ovotransferrin, ovalbumin, ovomucoid, β-parvalbumin, β-enolase, aldolase A, tropomyosin, collagen alpha, creatine kinase, triosephosphate isomerase, pyruvate kinase, β′-vitellogenin, PKM-like L-lactate dehydrogenase, glucose 6-phosphate isomerase, and glyceraldehyde3-phosphate dehydrogenase, shrimp tropomyosin Met e 1. Cha f 1, vicilins (7S globulins), 2S albumins, legumins (11S globulins), profilins, heveins, lipid transfer proteins, Ara h 1 through Ara h 17 inclusive, ω-5 gliadin, γ-35 secalin, γ-75 secalin, low-molecular-weight glutenin subunits (LMW-GS), C hordeins, 7-71 kD glycinins and conglycinins (Wilson et al. 2005).

Aeroallergenic eukaryotic cast-offs as sources for IgE mediated antigen protein can include multiple subunits, glycosylation, protein fragments of naturally occurring proteins with the proviso that antigenic portions of the naturally occurring are retained therein, and combinations thereof. Specific sources of aeroallergenic eukaryotic cast-offs include dust mite feces; pet danders, such as those of cats, dogs, and rodents. Specific food antigen proteins operative in the present invention illustratively include Der p 1 to Der p 23, Der f 1, Eur m 1, Fel d 1 to Fel d 8, Can f 1, Can f 2, and Mus m 1. It is appreciated that as these antigenic proteins are often inhaled, the IgE reaction thereto is often manifested clinically as asthma.

Insect venoms as sources for IgE mediated antigen protein can include multiple subunits, glycosylation, protein fragments of naturally occurring proteins with the proviso that antigenic portions of the naturally occurring venoms are retained therein, and combinations thereof. Specific sources of insect bite proteins include mosquitoes, horseflies, beetles, and fleas. Specific sources of insect venoms include the venoms of the bee, wasp, hornet, yellow jacket and ant. Specific food antigen proteins operative in the present invention illustratively include Aed a 1 to Aed a 4, Aed al 2, Aed al 3, Ano d 2, Cul q, Cul q 3, Tab y 1 to Tab y 5, Har a 1, Har a 2, Cte f 1 to Cte f 3. It is appreciated that as these antigenic proteins are often excreted into a blood sucking bite wound and as a result can be large protein up to 120 kD and cause dermatitis and systemic clinical manifestations.

The complexation of an antigenic protein to dIgA or sIgA in the present invention is performed by conventional techniques developed for IgA (Simon et al 2016, Elisela et al 2023). The food antigen prepared from the raw food may be in the form of a paste that is then dissolved in buffered saline and mixed with dIgA or secretory IgA in the proportions indicated previously such that the range of proportions described previously (see paragraph [0011] ) Another exemplary technique includes incubation at the desired ratio at 37° C. for a duration of from 1 to 20 hours to maximize binding. An exemplary complexation solution is 150 mM aqueous saline at a pH of from 7.0 to 7.5.

A formulation for administration to a subject includes at least dIgA or secretory IgA that is complexed with a first preselected allergen protein. In some inventive embodiments, formulation agents are also present to facilitate at least one of storage stability, or a dosing form of a liquid, a capsule, and an aerosol. In still other embodiments, a second composition that varies through the inclusion of a second preselected allergen protein in addition to the first preselected IgE mediated antigen protein, is also present in the formulation to target multiple antigen specific allergic responses in a subject.

A method for medical treatment of a subject involves the oral or nasal administration of a dIgA-or sIgA-allergen immune complex. The IgA constituent thereof is readily derived from a number of sources. One such source for polymeric IgA or dIgA is pooled human plasma following Cohn cold ethanol fractionation to produce fraction III precipitate as performed by those of skill in the art of protein separation. Another source is the strip solution derived from anion exchange chromatography columns which is used to recover IgG from pooled donor plasma. The IgA byproduct is either purified to dIgA or converted into secretory IgA by the addition of recombinant human secretory component and then complexed with its target allergen. The secretory IgA is further purified by adsorption onto a nickel column as performed by those of skill in the art of protein purification.

A more detailed description of isolation of an IgA component as a byproduct from pooled human plasma or hyperimmune pooled human plasma is as follows. Ethanol fractionation of pooled human plasma is a well-known process to prepare immunoglobulin G. Pooled human plasma is first obtained from licensed plasmapheresis centers in the United States and tested for various pathogens including the HIV virus. The first manufacturing step of commercial immunoglobulin G preparations may involve modified cold ethanol fractionation according to Cohn to produce Cohn fraction III preciptate which contains the otherwise unneeded polyclonal IgA. In the fractionation process, many infectious viruses are eliminated from the pooled human plasma. An alternative manufacturing step is adsorption onto an ion exchange medium. The adsorption step may selectively reduce the IgA concentration in the eluate to less than 0.1%. Such a step is important for producing immunoglobulin G for intravenous infusion into humans, since some individuals undergo an anaphylactic-like reaction if treated with intravenous IgG that contains IgA as an impurity. The polymeric IgA product is then removed from the ion exchange column by stripping the column with high molarity NaCl solution. The strip solution is then further processed to recover the IgA.

The modified cold ethanol fractionation process according to Cohn is a series of fractionations using various levels of ethanol, pH, and temperature to produce fraction II which is further treated to produce immunoglobulins as described above. In the fractionation method, pooled human plasma is first treated to produce a cryoprecipitate and cryo-supernatant. The cryo-supernatant is subjected to a first ethanol fractionation to yield a supernatant I. Supernatant I is subjected to a second ethanol fractionation to yield fraction II+III. Fraction II+III is subjected to a third ethanol fractionation procedure to yield a supernatant III and Fraction III precipitate. The fraction III precipitate enriched in IgA is generally discarded as an unwanted byproduct. According to embodiments of the invention, this unwanted IgA following purification is further treated by incubation with immobilized hydrolases to inactivate viruses and vasoactive substances. Such treatment has been proven to eliminate many viruses tested including HIV, Sindbis, and vaccinia. Other antiviral treatments, as known to those skilled in the art, are used in combination and consist of solvent detergent processes, nanofiltration, and/or heat inactivation. Usually, three antiviral steps are implemented. Following incubation to remove viruses, the concentration of the active material is adjusted with sterile saline or buffered solutions to ensure a constant amount of active material per milliliter of reconstituted product. Finally, the solution with a constant amount of reconstituted product is sterilized by filtration before use.

The ethanol fractionation process according to Cohn is well known in the art and is (Cohn et al. 1946, Oncley et al. 1949, and in most detail in pages 576-602, Kirk-Othmer Encyclopedia of Chemical Technology, Vol. 3, second edition (1963)). Alternatively, ion exchange chromatography may be used to obtain the dimeric and polymeric IgA byproduct during the manufacture of intravenous immunoglobulin. From 4% to 22% of plasma IgA is dimeric and polymeric IgA (Delacroix et al. 1981; Delacroix et al. 1983). The resulting dimeric and polymeric IgA-J chains are purified.

An alternative method for the purification of plasma IgG is anion exchange chromatography. The IgA remains on the column and can be recovered by “stripping” the column with 2 M NaCl. The strip solution contains IgA, IgG, and IgM as well as α macroglobulin, and a C3 complement protein. The solution is passed over immobilized conconavalin A which binds the IgM and C3 protein. The flowthrough contains the IgA. Histadine tagged recombinant human secretory component is added to the flowthrough, and binds to the dimeric and polymeric IgA forming secretory IgA. The solution is then passed through a nickel column. The histidine tagged recombinant human secretory component functions as an affinity tag for the entire secretory IgA molecule allowing its efficient industrial recovery in purified form, as detailed in U.S. Pat. No. 11,623,948 B2.

After the industrial recovery of the secretory IgA, the secretory IgA is mixed with the desired allergen protein to allow for formation of secretory IgA-allergen immune complex, sIgA-allergen, which is then used as a medication for a subject exhibiting allergic or sensitivity reactions to allergen.

Purified dimeric and polymeric IgA containing secretory component and complexed to the allergen, sIgA-allergen, is optionally formulated in glycine to concentrations of from 20-200 mg/mL.

Dimeric IgA contains two, IgA monomers per J chain, respectively.

The secretory IgA antibodies may be administered alone as a liquid. A liquid formulation is particularly well-suited for usage as a nasal aerosol or an inhaler or nebulized formulation as well as a swallowed medication.

Since preferred methods of administration are oral. These oral dosages are prepared according to conventional methods known by those skilled in the art. The secretory IgA antibodies may also be combined with other pharmaceutically acceptable carriers such as various liquids, including cow's milk. which may also provide additional nutritional and/or pharmaceutical benefits. Remington Science and Practice of Pharmacy, 20^{th} ed. (2000).

These compositions optionally contain adjuvants such as preserving, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the IgA can be brought about by the use of agents delaying absorption, for example, aluminum monostearate and gelatin.

Solid dosage forms for oral administration include capsules. In such solid dosage forms, the active compound is first lyophilized. It is re-constituted in a buffered water-based solvent.

Solid compositions of a similar type may also be employed as fillers in soft and hard-filled gelatin capsules using such excipients as lactose or milk sugar as well as high molecular weight polyethyleneglycols, and the like.

The solid dosage forms may contain opacifying agents and can also be of such composition that they release the active compound or compounds in a certain part of the intestinal tract in a delayed manner. Examples of embedding compositions which can be used are polymeric substances and waxes. The active compounds can also be in microencapsulated form, if appropriate, with one or more of the above-mentioned excipients.

Liquid dosage forms for ophthalmic, nasal, or oral administration include pharmaceutically acceptable emulsions, solutions, suspensions, syrups, and elixirs. In addition to the active compounds, the liquid dosage forms may contain inert diluents commonly used in the art, such as water, isotonic saline solution, or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

In some inventive embodiments, the effect of the secretory IgA antibody-allergen complexes is dependent on the complexes reaching the small intestine. The secretory component protects the molecule from digestion.

The amount of dIgA- or sIgA-allergen provided to the patient is about 600 gram per day for 5 days, for a total dose of up to 3 kg. Typically amounts from about 1 to 5 kg per day will be used and preferably, 5 to 10 grams per day. For example, about 5 grams of secretory IgA-food allergen protein could be given to a subject 1 to 2 times per day. The doses of the secretory IgA antibody-food allergen protein immune complex to be administered will depend upon the subject and the subject's medical history. Dosages of secretory IgA—food allergen protein for adult humans envisioned by the present invention and considered to be therapeutically effective will range from between about 5 kg to 500 mg depending upon whether the secretory IgA is polyclonal or monoclonal, respectively. However, it is to be understood that doses can readily be adjusted to provide appropriate amounts of the IgA antibody to any subject, including children.

The invention is further described by reference to the following detailed examples. These examples are not meant to limit the scope of the invention that has been set forth in the foregoing description. Variations within the concepts of the invention are apparent to those skilled in the art.

### EXAMPLE 1

Polyclonal IgA is obtained from pooled human plasma following Cohn cold ethanol fractionation to produce fraction III precipitate. IgA-J chain dimers and polymers converted to sIgA by the addition of histidine tagged recombinant human secretory component at a molar ratio of secretory component to IgA-J chain dimers and polymers of 1:1 and are purified by nickel affinity chromatography. The sIgA is further processed by the addition Ara h 1 peanut protein (Cabanos et al. 2011) as a food allergen protein at a molar ratio of 10:1 forming sIgA-allergen complex, sIgA-allergen. The sIgA-allergen is stabilized by the addition of glycine sufficient to prepare a 250 mM glycine solution. The final solution is adjusted to a total therapeutic dose of 3 kg secretory IgA-allergen immune complex administered in several smaller doses over 5 to 7 days. The sIgA-food allergen complex is administered to a person suffering with food allergy to peanuts. One month after initiation of treatment the food allergy sufferer eats the allergenic food without inducing any significant symptoms of an allergic reaction.

### EXAMPLE 2

The process of Example 1 is repeated with Ara h 3 (Cabanos et al. 2011) as the food allergen protein in place of Ara h 1. A similar result to Example 1 is noted.

### EXAMPLE 3

The compositions of Examples 1 and 2 are admixed in equal weight proportions and administered per Example 1. A similar result to Examples 1 and 2 is noted.

### EXAMPLE 4

As per Example 1, wherein the food allergen protein is peanut. sIgA-allergen possesses antigenic specificity to peanut and to suppress to anti-peanut IgE mediated mast cell activation (Konstantinou, et al. 2013, Simon et al. 2016).

### EXAMPLE 5

The process of Example 1 is repeated with dIgA in place of SIgA conjugated to Ara h 1 peanut protein with like clinical effect.

### REFERENCES

Berzofsky J. A., Berkower I. J., Epstein S. L., Monoclonal Antibodies in Chapter 12, Antigen-Antibody Interactions and Monoclonal Antibodies, pp. 455-465 in Fundamental Immunology, Third Edition, W. E. Paul (ed), Raven Press, NY 1993. Berzofsky et al., Fundamental Immunology, Third Edition, 1993; 455-462.
Cabanos C, Urabe H, Tandang-Silvas M R, Utsumi S, Mikami B, Maruyama N. Crystal structure of the major peanut allergen Ara h 1. Mol Immunol. 2011 October-November; 49(1-2):115-123.
Caubet J C, Nowak-Węgrzyn A. Current understanding of the immune mechanisms of food protein-induced enterocolitis syndrome. Expert Rev Clin Immunol. 2011 May; 7(3):317-27.
Chu D K, Wood R A, French S, Fiocchi A, Jordana M, Waserman S, Brożek J L, Schünemann H J. Oral immunotherapy for peanut allergy (PACE): a systematic review and meta-analysis of efficacy and safety. Lancet. 2019 Jun. 1; 393(10187):2222-2232.
Cohn E. J., Strong L. E., Hughes W. L., Jr., Mulford D. J., Ashworth J. N., Melin M., Taylor H. L., Preparation and Properties of Serum and Plasma Proteins IV. A System for the Separation into Fractions of the Protein and Lipoprotein Components of Biological Tissues and Fluids, J. Am. Chem. Soc. 1946; 68; 459-475.
Corthesy B., Recombinant Secretory IgA for Immune Intervention Against Mucosal Pathogens, Biochem. Soc. Trans. 1997, 25; 471-475.
Crottet P., Cottet S., Corthesy B., Expression, Purification and Biochemical Characterization of Recombinant Murine Secretory Component, A Novel Tool in Mucosal Immunology, Biochem. J. 1999, 341; 299-306.
Delacroix D. L., Hodgson H. J., McPherson A., Dive C., Vaerman J. P. Selective transport of polymeric immunoglobulin A in bile. Quantitative relationships of monomeric and polymeric immunoglobulin A, immunoglobulin M, and other proteins in serum, bile, and saliva. J. Clin. Invest. 1982 August; 70(2):230-241
Delacroix D. L., Elkom K. B., Geubel A. P., Hodgson H. F., Dive C., Vaerman J. P. Changes in size, subclass, and metabolic properties of serum immunoglobulin A in liver diseases and in other diseases with high serum immunoglobulin A. J. Clin. Invest. 1983 February; 71(2):358-367.
Fleischer D M, Greenhawt M, Sussman G, et al. Effect of Epicutaneous Immunotherapy vs Placebo on Reaction to Peanut Protein Ingestion Among Children With Peanut Allergy: The PEPITES Randomized Clinical Trial. JAMA. 2019; 321(10):946-955.
Frossard C P, Hauser C, Eigenmann P A. Antigen-specific secretory IgA antibodies in the gut are decreased in a mouse model of food allergy. J Allergy Clin Immunol. 2004 August; 114(2):377-382.
Granato D A, Piguet P F. A mouse monoclonal IgE antibody anti bovine milk beta-lactoglobulin allows studies of allergy in the gastrointestinal tract. Clin Exp Immunol. 1986 March; 63(3):703-710.
Jones R. M. L., Schweikart F., Frutiger S., Jaton J-C., Hughes G. J. Thiol-disulfide redox buffers maintain a structure of immunoglobulin A that is essential for optimal in vitro binding to secretory component. Biochimica et Biophysica Acta 1998; 1429:265-274.
Harrison M, Kilby A, Walker-Smith J A, France N E, Wood C B. Cows' milk protein intolerance: a possible association with gastroenteritis, lactose intolerance, and IgA deficiency. Br Med J. 1976 Jun. 19; 1(6024):1501-1504.
Kohler G., Milstein C., Continuous Cultures of Fused Cells Secreting Antibody of Predetermined Specificity, Nature 1975; 256; 495-497.
Konstantinou, G. N., Simon, M. R. Konstantinou, G. N., Simon, M. R., Gimenez, G., Bardina, L., Grishina, G., Sampson, H. A. Epitope recognition patterns of monomeric peanut-specific IgA in humans diverge significantly from dimeric peanut-specific IgA in human colostrum and human and mouse serum. Allergy 68 (Supplement s97):23-24, 2013. World Allergy Organization Congress, Milan, Italy, Jun. 23, 2013.
Lullau E., Heyse S., Vogel H., Marison I., von Stockar U., Kraehanbuhl J-P., Corthesy B., Antigen Binding Properties of Purified Immunoglulin A Antibodies, J. Biol. Chem. 1996; 271:16300-16309.
Marietta E, Black K, Camilleri M, Krause P, Rogers R S 3rd, David C, Pittelkow M R, Murray J A. A new model for dermatitis herpetiformis that uses HLA-DQ8 transgenic NOD mice. J Clin Invest. 2004 October; 114(8):1090-1097.
Matysiak-Budnik T, Moura I C, Arcos-Fajardo M, Lebreton C, Ménard S, Candalh C, Ben-Khalifa K, Dugave C, Tamouza H, van Niel G, Bouhnik Y, Lamarque D, Chaussade S, Malamut G, Cellier C, Cerf-Bensussan N, Monteiro R C, Heyman M. Secretory IgA mediates retrotranscytosis of intact gliadin peptides via the transferrin receptor in celiac disease. J Exp Med. 2008 Jan. 21; 205(1):143-154.
Oncley J. L., Melin M., Richert D. A., Cameron J. W., Gross P. M., Jr., The Separation of the Antibodies, Isoagglutinins, Prothrombin, Plasminogen and β1-Lipoprotein into Subfractions of Human Plasma. J. Am. Chem. Soc. 1949; 71:541-550.
The PALISADE Group of Clinical Investigators. AR101 Oral Immunotherapy for Peanut Allergy. N Engl J Med 2018; 379:1991-2001
Platts-Mills, T A E. “The role of immunoglobulin E in allergy and asthma.” American journal of respiratory and critical care medicine 164.supplement_1 (2001): S1-S5.
Possin M E, Morgan S, DaSilva D F, Tisler C, Pappas T E, Roberg K A, Anderson E, Evans M D, Gangnon R, Lemanske R F, Gern J E. The relationships among immunoglobulin levels, allergic sensitization, and viral respiratory illnesses in early childhood. Pediatr Allergy Immunol 2010: 21: 990-996.
Ra, Chisei, Marie-Hélène E. Jouvin, Ulrich Blank, and Jean-Pierre Kinet. “A macrophage Fc γ receptor and the mast cell receptor for IgE share an identical subunit.” Nature 341, no. 6244 (1989): 752-754.
Sampson, H A et al. Second symposium on the definition and management of anaphylaxis: Summary report—Second National Institute of Allergy and Infectious Disease/Food Allergy and Anaphylaxis Network Symposium. J Allergy Clin Immunol 2006; 117:391-397.
Schwarze J et al. Antigen-specific Immunoglobulin-A Prevents Increased Airway Responsiveness and Lung Eosinophilia after Airway Challenge in Sensitized Mice. AM J RESPIR CRIT CARE MED 1998; 158:519-525.
Shimoda M, Inoue Y, Azuma N, Kanno C. Local antibody response in Peyer's patches to the orally administered dietary protein antigen. Biosci Biotechnol Biochem. 1999 December; 63(12):2123-2129.
Simon, M. R., Konstantinou, G. N. Plasma derived IgA from healthy donors binds to peanut extract and inhibits peanut-induced rat basophil activation. J Allergy Clin Immunol 137(2, suppl):AB128, 2016.
Strong L. E., Blood Fractionation, pp. 576-602 in vol. 3, Kirk-Othmer Encyclopedia of Chemical Technology. Second Edition, H. F. Mark, J. J. McKetta, D. F. Othmer (eds), Interscience Publishers, NY 1963, pp. 576-602.
Symersky J., Novak J., McPherson D. T., DeLucas L., Mestecky J. Expression of the recombinant human immunoglobulin J chain in Escherichia coli. Mol. Immunol. 2000; 37:133-140.
Vighi G et al. Allergy and the gastrointestinal system. Clinical and Experimental Immunology, 153 (Suppl. 1): 3-6, 2008.
Vojdani A. Detection of IgE, IgG, IgA and IgM antibodies against raw and processed food antigens. Nutr Metab (Lond). 2009 May 12; 6:22
Walker A M, Kemp A S, Hill D J, and Shelton M J. Features of transient hypogammaglobulinaemia in infants screened for immunological abnormalities. Arch Dis Child. 1994 March; 70(3):183-186.
Wang M, Takeda K, Shiraishi Y, Okamoto M, Dakhama A, Joetham A, Gelfand E W. Peanut-induced intestinal allergy is mediated through a mast cell-IgE-Fcepsilon RI-IL-13 pathway. J Allergy Clin Immunol. 2010 August; 126(2):306-16, 316.e1-12.
Wilson, S.; Blaschek, K.; Mejia, E. G. Allergenic proteins in soybean: processing and reduction of P34 allergenicity. J. Nutr. Rev., 2005, 63 (2), 47-58
Wong, Y. W., Kussie, P. H., Parhamiseren, B. & Margolies, M. N. (1995). Modulation of antibody affinity by an engineered amino-acid substitution. J. Immunol. 154, 3351-3358.

Patent applications and publications mentioned in the specification are indicative of the levels of those skilled in the art to which the invention pertains. These applications and publications are incorporated herein by reference to the same extent as if each individual application or publication was specifically and individually incorporated herein by reference.

The foregoing description is illustrative of particular embodiments of the invention, but is not meant to be a limitation upon the practice.

The following claims, including all equivalents thereof, are intended to define the scope of the invention.

1. A composition comprising:
dimeric or secretory immunoglobin A;
an allergen protein, the allergen protein complexed to the dimeric or secretory immunoglobin A.

2. The composition of claim 1 wherein the secretory immunoglobin A is present and comprises polyclonal IgA-J chain dimers or IgA-J chain polymers are combined with a recombinant secretory component in a molar ratio of the IgA-J chain dimers or IgA-J chain polymers to the recombinant secretory component of 1:1.

3. The composition of claim 1 wherein the secretory immunoglobin A and the allergen protein are present in a molar ratio of 4000 to 4:1.

4. The composition of claim 2 wherein the polyclonal IgA-J chain dimers or IgA-J chain polymers are combined with said recombinant secretory component by a disulfide linkage.

5. The composition of claim 2 wherein the polyclonal IgA-J chain dimers or IgA-J chain polymers are derived from specific allergen immune donors.

6. The composition of claim 1 wherein the allergen protein comprises multiple subunits.

7. The composition of claim 1 wherein the allergen protein is glycosylated.

8. The composition of claim 1 wherein the allergen protein comprises a protein fragment of a naturally occurring protein with the proviso that an antigenic epitope of the naturally occurring protein is retained.

9. The composition of claim 1 wherein the allergen protein comprises a cow's milk protein, a chicken egg protein, a fish protein, a crustacean protein, a tree nut protein, a peanut protein, a wheat protein, a legume protein, or a sesame protein.

10. The composition of claim 1 wherein the allergen protein is casein, β-lactoglobulin, α-lactalbumin, ovotransferrin, ovalbumin, ovomucoid, β-parvalbumin, β-enolase, aldolase A, tropomyosin, collagen alpha, creatine kinase, triosephosphate isomerase, pyruvate kinase, β′-vitellogenin, PKM-like L-lactate dehydrogenase, glucose 6-phosphate isomerase, glyceraldehyde3-phosphate dehydrogenase, shrimp tropomyosin Met e 1, Cha f 1, a vicilin, a 2S albumin, a legumin, a profilin, a hevein, a lipid transfer protein, Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13, Ara h 14, Ara h 15, Ara h 16, Ara h 17, ω-5 gliadin, γ-35 secalin, γ-75 secalin, a low-molecular-weight glutenin subunits), a C hordein, a glycinin, a conglycinin, Hymenoptera insect sting venom, or a combination thereof.

11. The composition of claim 1 wherein the allergen protein derived from dust mite feces, a pet dander, or insect venom.

12. The composition of claim 1 wherein the dimeric immunoglobin A is present.

13. A formulation comprising:
a composition of claim 1 with one of the allergen proteins that is lyophilized.

14. The formulation of claim 13 in a form of a tablet or a capsule.

15. A process for treating IgE mediated allergy in a human comprising:
administering a therapeutically effective amount of a composition comprising: dimeric or secretory immunoglobin A; and an allergen protein, the allergen protein complexed to the dimeric or secretory immunoglobin A.

16. The process of claim 15 wherein the therapeutically effective amount is between 3 grams and 3 kilograms per 60 kg recipient.

17. The process of claim 15 wherein the therapeutically effective amount is between 3 milligrams and 3 grams per 60 kg recipient when administered as an inhaled solution.
